SEQUENCE LISTING (11 sequences)

SEQ ID NO: 1 - DNA, 4711 bp, Homo sapiens
agtgagctatggggacactactgcactgtagcctgggcaacagagcaagaccttgtctcaaaaatgtatatatattttgggctttttttcctaaaacgggaactacaacagcatatttgcgagctgatgagagtgacccagcagagagggaaatggatcagctctgttgaagatgcactggacaccagaacacgcccagcccctcaaccagtggccagagcagcacctggacgtctcctccaccaccccgtcgccggcccacaagttggagttgccccctgggggtcgccaacgctgccactacgcttgggcacacgacgacatctcagccctcactgcctccaacctcctaaagcgctatgcagagaagtactctggggtcttggattctccctacgagcgtccggccctgggcgggtacagcgacgcctccttcctcaacggcgccaaaggggatcccgagccctggccagggccggagccaccctaccccttggcctcactccacgaaggcctcccaggaaccaaatcgggcggtggcggcggttccggggccctggggggctccccagttttagccgggaacctccctgaacccctctacgccggcaatgcgtgcgggggcccatcggcggcgcccgagtacgcggccggctacggcggggggtacctggcgccgggttactgcgcgcagacgggcgccgcgctgcccccgccgcccccggccgcgctcctgcagcccccaccgcctccggggtacgggccctcagcgccgctgtacaactatcccgcagggggctacgcagcgcagcccggctatggcgcgctcccgccgcccccaggcccacccccggccccctacctgaccccgggcctgcccgcgcccacgcccctgcccgcgccggcaccgcccaccgcctatggcttccccacggccgcgccgggtgccgaatccgggctgtcgctgaagcgcaaggccgccgacgaggggcccgagggccgctaccgcaagtacgcgtacgagcccgccaaggcccccgtggctgacggagcctcctaccccgccgcggacaacggcgaatgtcggggcaacgggttccgggccaagccgccaggagccgcggaggaggcgtcgggcaagtacggtggcggcgtccccctcaaggtcctgggctcccccgtctacggcccgcaactggagccctttgaaaagttcccggagcgggccccggctcctcgtggggggttcgccgtgccgtcgggggagactcccaaaggcgtggaccctggggccctggagctggtgacgagcaagatggtggactgcgggcccccggtgcagtgggcggatgtggcgggccagggcgcgctcaaggcggcgctggaggaggagctggtgtggcccctgctcaggccgcccgcctacccgggcagcctgcgcccgccgcggaccgtcctgctctttgggccgcggggcgcgggcaaagcgctgctgggccgctgcctcgccacgcagctgggcgccacgctgttgcgcctgcgcggcgcgaccctggctgcgcccggcgccgccgagggcgcgcgcctcctccaggccgccttcgcggccgcgcgctgccgcccaccctccgtactcctcatcagcgagctagaggcgctgctccccgcccgggacgacggcgcggcggcagggggcgcgctgcaggtgccgctcctggcctgcctggacgggggctgcggcgcgggggctgacggcgtgctggttgtgggcaccacctcgcggcccgcggctctggacgaggcgacccgccggcgcttctctctccgcttctacgtggcgctgcccgacagcccggcccgcgggcagatcctgcagcgggcgctggcccagcagggctgcgcgctcagtgagcgggaactggcggcgctggtgcagggcacgcagggcttctctgggggcgagctggggcagctgtgccagcaggcggcggccggggcgggcctcccggggctgcagcgccccctctcctacaaggacctggaggcggcgctggccaaggtgggccctagggcctctgccaaggaactggactcgttcgtggagtgggacaaaatgtacggctccggacactgacggcgcgcgggggaggccgcgggagccgcagtccctccgtccccgccgcctccgcgtgggagggatgtcactgactaaacccggctggcaggggctggagtggtgaatgtgggatcggggacaggaggggtctgccggtggatattttttttttcgtgggaaggaaaatgcttctgccaggcagatgccatatgcgccgtgtactcaggtttttcctatttattgtggactggaagctcgccatctccgcccggcagaccgggcagatccggcatgggctggcacccggggccttaagaactcctgctctcttgccacaacgcttttgtctcctcgctatctgaatggcaccctccttctccctcactctctccatcccattctctgcattctcttggttttctctcccttttgctttgtcgctgacacccctgcccaccccatgctggccctgtttctctcctgcccctccctccccagctctccatccctcaccctctgtgcttctgtctccatccctggctctccagcgtccctggccttttggtccctgagctttaatgcctttccctgccttctgttcttatttggactgcagtggccctttgcaggagctctggaggcccaggggctgaggaggagggttacccctctacccatctgaaacctagggtctagggggatcaaggaaaaaaagtccccaaagaaggggaattttttgtttgtttttgaggggagatcccagaaatgtagcttgtttcatattttagtcttcttatttttgtaaaatgtgtagaatttgctgtttttctttttcttttgacaactcaggaagaaactgacctcagaaagaatgttagactttggctgctctcctgtgtgcccctcacacctgccccctcccccccactccatccaggggaccaaattctcccagacactcaaaaaatgagacttacggggaaggggagaggaagacccagaggcctcagtgaaaccccagctattcctggtcagaagcagaatgtattcctaagggcttcctccccagggccgaggcctaggcatgaatgtggggagtgggctgtggggtttgagagaagggaggccttattcctctcctgctgctccccaccccctgccccacccaacccctccgctgagtgttttctgtgaagggctatccagagttaggatgcccttgcccaattccttcctgagacccagaaggtagggtgggagggcccaaatgggaaggtgacctaagcagaaagtctccagaaaggtcatgtcccctggccctgccttggcagaggtccccagtgacttatgctaggaggattccatctgggtagacagtctggccacaaaatcagctactggacctcagccatctctgctggaggctctgaggaggagtgagcatccctcacttgtgggggctctgtgaggaaatgtgccttccccattcccccggagtcctaggtctggagctccagggctgggagagggtgagggagatgggcaggggtgttttctctgaccttgggggcttagtctcagtcctgcctgaactttccactaggcttggaacccttccaagaaccatatttctctccttcccaccaattttcccttgatgaggctttagcagtttgctcccaccacccccagcccatttcacaactctgatcttagtccaaagcaggggacacgcccccccaccaccactttttctctctcccatctcagcctcctgtgcagttccttgcctgcccgtgcatttcctagagtctactgcctcccccctggctgggagggtgtctgggggggatctttcaggggccctggcacccagggcctgtgctggcctaggagtgctgaccagaaggctgctctgttcccccccacccccgttgctttctggccccctctttggagccagccacccacagggctttggtgcctcagaagcagtgggctgccgggtcacagccgcaggctgcaaaagaccctcggagggagcatggagtgaggggttctctctcaggtgtgtatgtattggggggtgggggtgggtggagggtgtcagggaagttggggtgggatcccagccttcccttcaagaggcagggagctctgggaggtggagtccccaccgctttctctactaggctcctcctgttccccaggcttggggagctttgcacaaggagactgcccccagcctagtggcacctacctcatgggctctggggcaggtaggggaagggccagtccagctctggtaatgctggggggaggcataccaaagaatccaggggcagggagtggggagggtgacttccgagctggcctctccccttcctctacccagactggggctgggatcctctcctcccgctgtaaccatttctacctcattttgctgcgtgttgtacatggacgtatttatctcctgtctgacgatgctctgcagttgtggtctgtctacctcagaagagactgtattttaaaagaaagtattacacagtattaaagcgatgacatgtggtttgcaaaaaaaaaaaaaaaaaa

SEQ ID NO: 2 - protein, 653 aa, Homo sapiens
MHWTPEHAQPLNQWPEQHLDVSSTTPSPAHKLELPPGGRQRCHYAWAHDDISALTASNLLKRYAEKYSGVLDSPYERPALGGYSDASFLNGAKGDPEPWPGPEPPYPLASLHEGLPGTKSGGGGGSGALGGSPVLAGNLPEPLYAGNACGGPSAAPEYAAGYGGGYLAPGYCAQTGAALPPPPPAALLQPPPPPGYGPSAPLYNYPAGGYAAQPGYGALPPPPGPPPAPYLTPGLPAPTPLPAPAPPTAYGFPTAAPGAESGLSLKRKAADEGPEGRYRKYAYEPAKAPVADGASYPAADNGECRGNGFRAKPPGAAEEASGKYGGGVPLKVLGSPVYGPQLEPFEKFPERAPAPRGGFAVPSGETPKGVDPGALELVTSKMVDCGPPVQWADVAGQGALKAALEEELVWPLLRPPAYPGSLRPPRTVLLFGPRGAGKALLGRCLATQLGATLLRLRGATLAAPGAAEGARLLQAAFAAARCRPPSVLLISELEALLPARDDGAAAGGALQVPLLACLDGGCGAGADGVLVVGTTSRPAALDEATRRRFSLRFYVALPDSPARGQILQRALAQQGCALSERELAALVQGTQGFSGGELGQLCQQAAAGAGLPGLQRPLSYKDLEAALAKVGPRASAKELDSFVEWDKMYGSGH

SEQ ID NO: 3 - DNA, 21 bp, Homo sapiens
uuacacaguauuaaagcgauu

SEQ ID NO: 4 - DNA, 21 bp, Homo sapiens
ucgcuuuaauacuguguaauu

SEQ ID NO: 5 - DNA, 21 bp, Homo sapiens
caucugaaaccuagggucuuu

SEQ ID NO: 6 - DNA, 21 bp, Homo sapiens
agacccuagguuucagauguu

SEQ ID NO: 7 - DNA, 21 bp, Homo sapiens
gugacuuaugcuaggaggauu

SEQ ID NO: 8 - DNA, 21 bp, Homo sapiens
uccuccuagcauaagucacuu

SEQ ID NO: 9 - DNA, 21 bp, Homo sapiens
ggucagaagcagaauguauuu

SEQ ID NO: 10 - DNA, 21 bp, Homo sapiens
auacauucugcuucugaccuu

SEQ ID NO: 11 - protein, 472 aa, Homo sapiens
MTTCSRQFTSSSSMKGSCGIGGGIGGGSSRISSVLAGGSCRAPSTYGGGLSVSSSRFSSGGACGLGGGYGGGFSSSSSSFGSGFGGGYGGGLGAGLGGGFGGGFAGGDGLLVGSEKVTMQNLNDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

(19) United States
(12) Patent Application Publication
(10) Pub. No.: US 2020/0289394 A1
(43) Pub. Date: Sep. 17, 2020

(54) METHOD FOR RESTORING HAIR FOLLICLES AND HAIR GROWTH

(71) Applicant: MICROCURES, INC., Santa Cruz, CA (US)

(72) Inventor: Brian O'ROURKE, Rockville Centre, NY (US)

(73) Assignee: MICROCURES, INC., Santa Cruz, CA (US)

(21) Appl. No.: 16/768,398

(22) PCT Filed: Oct. 23, 2018
(86) PCT No.: PCT/US2018/056972

§ 371 Date: May 29, 2020

Related U.S. Application Data
(60) Provisional application No. 62/592,566, filed on Nov. 30, 2017.

(51) Int. Cl. A61K 8/60; A61K 31/712; A61K 8/46; A61K 8/73

(52) U.S. Cl. CPC A61K 8/606; A61K 31/712; A61Q 7/00; A61K 8/735; A61K 8/463

(57) ABSTRACT

The disclosure provides methods of enhancing hair growth and hair follicle restoration using a siRNA or shRNA directed against a human Fidgetin like-2 nucleic acid. Specifically, the disclosure provides compositions comprising siRNA or shRNA against a human Fidgetin like-2 nucleic acid, and the methods of using the compositions. Further disclosed are siRNA sequences against a human Fidgetin like-2 nucleic acid and the formulations of the compositions.

### CROSS-REFERENCE TO RELATED APPLICATIONS

This application claims benefit of U.S. Provisional Application No. 62/592,566, filed Nov. 30, 2017, the contents of which are hereby incorporated by reference.

### BACKGROUND OF THE INVENTION

The disclosures of all publications, patents, patent application publications and books referred to in this application are hereby incorporated by reference in their entirety into the subject application to more fully describe the art to which the subject invention pertains.

Hair loss is a major esthetic and cosmetic concern to both men and women. Improved methods to restore hair follicles and/or increase hair growth are desirable.

The present invention addresses this need.

### SUMMARY OF THE INVENTION

A method is provided for enhancing hair follicle growth in skin comprising directly administering to the skin an amount of a siRNA or shRNA directed against a DNA or RNA encoding a human Fidgetin like-2 effective to enhance hair follicle growth in skin.

Also provided is a method for increasing hair growth in skin comprising directly administering to the skin an amount of a siRNA or shRNA directed against a DNA or RNA encoding a human Fidgetin like-2 effective to increase hair growth in skin.

Also provided is a method for increasing Cytokeratin 14 expression in skin comprising directly administering to the skin an amount of a siRNA or shRNA directed against a DNA or RNA encoding a human Fidgetin like-2 effective to increase Cytokeratin 14 expression in skin.

A shampoo composition is provided comprising (i) an amount of a siRNA or shRNA directed against a DNA or RNA encoding a human Fidgetin like-2 effective to enhance hair follicle growth in skin and (ii) a surfactant.

### BRIEF DESCRIPTION OF FIGURES

FIG. 1. Poloxamer FL2-siRNA was applied to mouse skin portion from which hair follicles have been removed. Representative H&E staining of bisected skin sections for Control-siRNA and FL2-siRNA groups taken at 8 days. Epithelial hyperplasia is present in the epidermis of the control-siRNA group while FL2-siRNA treated animals show cytokeratin 14 positive hair follicles within the portion from which hair follicles have been removed. Double-headed arrows designate observable areas initially treated to remove follicles.

FIG. 2. 2-photon confocal microscopy imaging shows nanoparticle encapsulated FL2 siRNA enhances hair follicle formation in skin previously treated to remove hair follicles.

FIG. 3. FL2 siRNA stimulates the restoration of hair follicles to bum wound sites. Animal skin was subjected to third degree thermal bums under anesthesia and then topically treated with FL2-siRNA. Following sacrifice on day 56, wounds were sectioned, stained with H&E and examined for adnexal structures. Hair follicles (indicated by the black arrow) were noted within the wound zone in FL2 siRNA treated conditions, while none were present in controls.

### DETAILED DESCRIPTION OF THE INVENTION

A method is provided for enhancing hair follicle growth in skin comprising directly administering to the skin an amount of a siRNA or shRNA directed against a DNA or RNA encoding a human Fidgetin like-2 effective to enhance hair follicle growth in skin.

Also provided is a method for increasing hair growth in skin comprising directly administering to the skin an amount of a siRNA or shRNA directed against a DNA or RNA encoding a human Fidgetin like-2 effective to increase hair growth in skin.

Also provided is a method for increasing Cytokeratin 14 expression in skin comprising directly administering to the skin an amount of a siRNA or shRNA directed against a DNA or RNA encoding a human Fidgetin like-2 effective to increase Cytokeratin 14 expression in skin.

In embodiments, the Fidgetin like-2 comprises the amino acid set forth in SEQ ID NO:2

In embodiments, the siRNA is administered.

In embodiments, the shRNA is administered.

In embodiments, the siRNA directed against a DNA or RNA encoding human Fidgetin-like 2 has at least one 2′ sugar modification.

In embodiments, the shRNA directed against a DNA or RNA encoding human Fidgetin-like 2 has at least one 2′ sugar modification.

In embodiments, the siRNA or shRNA is directed against an mRNA encoding the human Fidgetin-like 2.

In embodiments, the siRNA or shRNA is directed against an DNA encoding the human Fidgetin-like 2.

In embodiments, the siRNA comprises a sequence set forth in SEQ ID NOS:3, 4, 5, 6, 7, 8, 9, or 10.

In an embodiment of the methods, the siRNA comprises a sequence set forth in SEQ ID NOS:3, 4, 5, 6, 7, 8, 9, or 10.

In an embodiment, the Fidgetin like-2 comprises the amino acid set forth in SEQ ID NO:2. In an embodiment, the siRNA is administered. In an embodiment, the shRNA is administered. In an embodiment, the siRNA directed against a DNA or RNA encoding human Fidgetin-like 2 has at least one 2′ sugar modification. In an embodiment, the shRNA directed against a DNA or RNA encoding human Fidgetin-like 2 has at least one 2′ sugar modification. In an embodiment, the siRNA or shRNA is directed against an mRNA encoding the human Fidgetin-like 2. In an embodiment, the siRNA or shRNA is directed against an DNA encoding the human Fidgetin-like In an embodiment, the siRNA comprises a sequence set forth in SEQ ID NOS:3, 4, 5, 6, 7, 8, 9, or 10.

In an embodiment, siRNA or shRNA administration is effected by administering liposomes containing the siRNA or shRNA. In an embodiment, siRNA or shRNA administration is effected by administering a nanoparticle encapsulating the siRNA or shRNA. In an embodiment, siRNA or shRNA administration is effected by administering a poloaxomer-comprising nanoparticle encapsulating the siRNA or shRNA.

In an embodiment, the inhibitor of Fidgetin-like 2 is a siRNA directed against a DNA or RNA encoding a human Fidgetin like-2. In an embodiment, the inhibitor of Fidgetin-like 2 is a shRNA directed against a DNA or RNA encoding a human Fidgetin like-2.

In an embodiment, the inhibitor of Fidgetin-like 2 is administered topically to the skin. In an embodiment, the inhibitor of Fidgetin-like 2 is administered from a reservoir that elutes the inhibitor, for example an eluting skin patch. In an embodiment, the inhibitor of Fidgetin-like 2 is administered from microneedle patch, wherein the miconeedles deliver the inhibitor of Fidgetin-like 2, such as the siRNA, into the skin when placed on the skin or adhered onto the skin.

In an embodiment, the Fidgetin-like 2 is human Fidgetin-like 2.

The dosage of the inhibitor administered in treatment will vary depending upon factors such as the pharmacodynamic characteristics of a specific inhibitor and its mode and route of administration; the age, sex, metabolic rate, absorptive efficiency, health and weight of the recipient; the nature and extent of the symptoms; the kind of concurrent treatment being administered; the frequency of treatment with the inhibitor and the desired therapeutic effect.

A dosage unit of the inhibitor may comprise a single compound, or a mixture of the compound with, for example, one or more hair growth-stimulating compound(s).

In an embodiment, the siRNA (small interfering RNA) as used in the methods or compositions described herein comprises a portion which is complementary to an mRNA sequence encoding a Fidgetin-like 2 protein. In an embodiment, the Fidgetin-like 2 protein is a human Fidgetin-like 2 protein. In an embodiment, the mRNA is encoded by the DNA sequence NCBI Reference Sequence: NM_001013690.4 (SEQ ID NO:1), and the siRNA is effective to inhibit expression of Fidgetin-like 2 protein. In an embodiment, the Fidgetin-like 2 protein comprises consecutive amino acid residues having the sequence set forth in SEQ ID NO:2.

In an embodiment, the siRNA comprises a double-stranded portion (duplex). In an embodiment, the siRNA is 19-25 nucleotides in length. In an embodiment, the siRNA is 20-25 nucleotides in length. In an embodiment the siRNA comprises a 19-21 core RNA duplex with a one or two nucleotide 3′ overhang on, independently, either one or both strands. In an embodiment the siRNA comprises a 19-25 RNA duplex with a one or two nucleotide 3′ overhang on, independently, either one or both strands. The siRNA can be 5′ phosphorylated, or not, and may be modified with any of the known modifications in the art to improve efficacy and/or resistance to nuclease degradation. In an embodiment the siRNA can be administered such that it is transfected into one or more cells. In an embodiment, the siRNA is 5′ phosphorylated. In an embodiment, the whole length of the non-overlapping portion of the siRNA is fully complementary to a portion of a mRNA encoding a Fidgetin-like 2 protein.

In an embodiment, the 5′ terminal residue of a strand of the siRNA is phosphorylated. In an embodiment the 5′ terminal residue of the antisense strand of the siRNA is phosphorylated. In one embodiment, a siRNA of the invention comprises a double-stranded RNA wherein one strand of the double-stranded RNA is 80, 85, 90, 95 or 100% complementary to a portion of an RNA transcript of a gene encoding Fidgetin-like 2 protein. In an embodiment, the RNA transcript of a gene encoding Fidgetin-like 2 protein is an mRNA. In an embodiment, the Fidgetin-like 2 protein is a human Fidgetin-like 2 protein. In an embodiment, a siRNA of the invention comprises a double-stranded RNA wherein one strand of the RNA comprises a portion having a sequence the same as a portion of 18-25 consecutive nucleotides of an RNA transcript of a gene encoding Fidgetin-like 2 protein. In an embodiment, the Fidgetin-like 2 protein is a human Fidgetin-like 2 protein. In yet another embodiment, a siRNA of the invention comprises a double-stranded RNA wherein both strands of RNA are connected by a non-nucleotide linker. Alternately, a siRNA of the invention can comprise a double-stranded RNA wherein both strands of RNA are connected by a nucleotide linker, such as a loop or stem loop structure. In an embodiment, both of the strands of RNA are not connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length.

In another embodiment, an siRNA of the invention comprises at least one 2′-sugar modification. In another embodiment, an siRNA of the invention comprises at least one nucleic acid base modification. In another embodiment, an siRNA of the invention comprises at least one phosphate backbone modification. In embodiments, an siRNA of the invention comprises at least one 2′-O-methyl modification. In embodiments, an siRNA of the invention comprises at least one phosphorodithioate (PS2).

As used herein, “at least one” means one or more.

In one embodiment, RNAi inhibition of Fidgetin-like 2 protein is effected by a short hairpin RNA (“shRNA”). The shRNA can be introduced into the appropriate cell by transduction with a vector. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene/mRNA, in the present case the mRNA encodes Fidgetin-like 2 protein. In an embodiment the Fidgetin-like 2 protein is a human Fidgetin-like 2 protein. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU.

In an embodiment, the FL2 is encoded by NCBI Reference Sequence:

| NM_001013690.4 (SEQ ID NO: 1) (nucleic acid encoding Human | |
|---|---|
| Fidgetin-like 2) | |
| 1 | agtgagctat ggggacacta ctgcactgta gcctgggcaa cagagcaaga ccttgtctca |
| 61 | aaaatgtata tatattttgg gctttttttc ctaaaacggg aactacaaca gcatatttgc |
| 121 | gagctgatga gagtgaccca gcagagaggg aaatggatca gctctgttga agatgcactg |
| 181 | gacaccagaa cacgcccagc ccctcaacca gtggccagag cagcacctgg acgtctcctc |
| 241 | caccaccccg tcgccggccc acaagttgga gttgccccct gggggtcgcc aacgctgcca |
| 301 | ctacgcttgg gcacacgacg acatctcagc cctcactgcc tccaacctcc taaagcgcta |
| 361 | tgcagagaag tactctgggg tcttggattc tccctacgag cgtccggccc tgggcgggta |
| 421 | cagcgacgcc tccttcctca acggcgccaa aggggatccc gagccctggc cagggccgga |
| 481 | gccaccctac cccttggcct cactccacga aggcctccca ggaaccaaat cgggcggtgg |
| 541 | cggcggttcc ggggccctgg ggggctcccc agttttagcc gggaacctcc ctgaacccct |
| 601 | ctacgccggc aatgcgtgcg ggggcccatc ggcggcgccc gagtacgcgg ccggctacgg |
| 661 | cggggggtac ctggcgccgg gttactgcgc gcagacgggc gccgcgctgc ccccgccgcc |
| 721 | cccggccgcg ctcctgcagc ccccaccgcc tccggggtac gggccctcag cgccgctgta |
| 781 | caactatccc gcagggggct acgcagcgca gcccggctat ggcgcgctcc cgccgccccc |
| 841 | aggcccaccc ccggccccct acctgacccc gggcctgccc gcgcccacgc ccctgcccgc |
| 901 | gccggcaccg cccaccgcct atggcttccc cacggccgcg ccgggtgccg aatccgggct |
| 961 | gtcgctgaag cgcaaggccg ccgacgaggg gcccgagggc cgctaccgca agtacgcgta |
| 1021 | cgagcccgcc aaggcccccg tggctgacgg agcctcctac cccgccgcgg acaacggcga |
| 1081 | atgtcggggc aacgggttcc gggccaagcc gccaggagcc gcggaggagg cgtcgggcaa |
| 1141 | gtacggtggc ggcgtccccc tcaaggtcct gggctccccc gtctacggcc cgcaactgga |
| 1201 | gccctttgaa aagttcccgg agcgggcccc ggctcctcgt ggggggttcg ccgtgccgtc |
| 1261 | gggggagact cccaaaggcg tggaccctgg ggccctggag ctggtgacga gcaagatggt |
| 1321 | ggactgcggg cccccggtgc agtgggcgga tgtggcgggc cagggcgcgc tcaaggcggc |
| 1381 | gctggaggag gagctggtgt ggcccctgct caggccgccc gcctacccgg gcagcctgcg |
| 1441 | cccgccgcgg accgtcctgc tctttgggcc gcggggcgcg ggcaaagcgc tgctgggccg |
| 1501 | ctgcctcgcc acgcagctgg gcgccacgct gttgcgcctg cgcggcgcga ccctggctgc |
| 1561 | gcccggcgcc gccgagggcg cgcgcctcct ccaggccgcc ttcgcggccg cgcgctgccg |
| 1621 | cccaccctcc gtactcctca tcagcgagct agaggcgctg ctccccgccc gggacgacgg |
| 1681 | cgcggcggca gggggcgcgc tgcaggtgcc gctcctggcc tgcctggacg ggggctgcgg |
| 1741 | cgcgggggct gacggcgtgc tggttgtggg caccacctcg cggcccgcgg ctctggacga |
| 1801 | ggcgacccgc cggcgcttct ctctccgctt ctacgtggcg ctgcccgaca gcccggcccg |
| 1861 | cgggcagatc ctgcagcggg cgctggccca gcagggctgc gcgctcagtg agcgggaact |
| 1921 | ggcggcgctg gtgcagggca cgcagggctt ctctgggggc gagctggggc agctgtgcca |
| 1981 | gcaggcggcg gccggggcgg gcctcccggg gctgcagcgc cccctctcct acaaggacct |
| 2041 | ggaggcggcg ctggccaagg tgggccctag ggcctctgcc aaggaactgg actcgttcgt |
| 2101 | ggagtgggac aaaatgtacg gctccggaca ctgacggcgc gcgggggagg ccgcgggagc |
| 2161 | cgcagtccct ccgtccccgc cgcctccgcg tgggagggat gtcactgact aaacccggct |
| 2221 | ggcaggggct ggagtggtga atgtgggatc ggggacagga ggggtctgcc ggtggatatt |
| 2281 | ttttttttcg tgggaaggaa aatgcttctg ccaggcagat gccatatgcg ccgtgtactc |
| 2341 | aggtttttcc tatttattgt ggactggaag ctcgccatct ccgcccggca gaccgggcag |
| 2401 | atccggcatg ggctggcacc cggggcctta agaactcctg ctctcttgcc acaacgcttt |
| 2461 | tgtctcctcg ctatctgaat ggcaccctcc ttctccctca ctctctccat cccattctct |
| 2521 | gcattctctt ggttttctct cccttttgct ttgtcgctga cacccctgcc caccccatgc |
| 2581 | tggccctgtt tctctcctgc ccctccctcc ccagctctcc atccctcacc ctctgtgctt |
| 2641 | ctgtctccat ccctggctct ccagcgtccc tggccttttg gtccctgagc tttaatgcct |
| 2701 | ttccctgcct tctgttctta tttggactgc agtggccctt tgcaggagct ctggaggccc |
| 2761 | aggggctgag gaggagggtt acccctctac ccatctgaaa cctagggtct agggggatca |
| 2821 | aggaaaaaaa gtccccaaag aaggggaatt ttttgtttgt ttttgagggg agatcccaga |
| 2881 | aatgtagctt gtttcatatt ttagtcttct tatttttgta aaatgtgtag aatttgctgt |
| 2941 | ttttcttttt cttttgacaa ctcaggaaga aactgacctc agaaagaatg ttagactttg |
| 3001 | gctgctctcc tgtgtgcccc tcacacctgc cccctccccc ccactccatc caggggacca |
| 3061 | aattctccca gacactcaaa aaatgagact tacggggaag gggagaggaa gacccagagg |
| 3121 | cctcagtgaa accccagcta ttcctggtca gaagcagaat gtattcctaa gggcttcctc |
| 3181 | cccagggccg aggcctaggc atgaatgtgg ggagtgggct gtggggtttg agagaaggga |
| 3241 | ggccttattc ctctcctgct gctccccacc ccctgcccca cccaacccct ccgctgagtg |
| 3301 | ttttctgtga agggctatcc agagttagga tgcccttgcc caattccttc ctgagaccca |
| 3361 | gaaggtaggg tgggagggcc caaatgggaa ggtgacctaa gcagaaagtc tccagaaagg |
| 3421 | tcatgtcccc tggccctgcc ttggcagagg tccccagtga cttatgctag gaggattcca |
| 3481 | tctgggtaga cagtctggcc acaaaatcag ctactggacc tcagccatct ctgctggagg |
| 3541 | ctctgaggag gagtgagcat ccctcacttg tgggggctct gtgaggaaat gtgccttccc |
| 3601 | cattcccccg gagtcctagg tctggagctc cagggctggg agagggtgag ggagatgggc |
| 3661 | aggggtgttt tctctgacct tgggggctta gtctcagtcc tgcctgaact ttccactagg |
| 3721 | cttggaaccc ttccaagaac catatttctc tccttcccac caattttccc ttgatgaggc |
| 3781 | tttagcagtt tgctcccacc acccccagcc catttcacaa ctctgatctt agtccaaagc |
| 3841 | aggggacacg cccccccacc accacttttt ctctctccca tctcagcctc ctgtgcagtt |
| 3901 | ccttgcctgc ccgtgcattt cctagagtct actgcctccc ccctggctgg gagggtgtct |
| 3961 | gggggggatc tttcaggggc cctggcaccc agggcctgtg ctggcctagg agtgctgacc |
| 4021 | agaaggctgc tctgttcccc cccacccccg ttgctttctg gccccctctt tggagccagc |
| 4081 | cacccacagg gctttggtgc ctcagaagca gtgggctgcc gggtcacagc cgcaggctgc |
| 4141 | aaaagaccct cggagggagc atggagtgag gggttctctc tcaggtgtgt atgtattggg |
| 4201 | gggtgggggt gggtggaggg tgtcagggaa gttggggtgg gatcccagcc ttcccttcaa |
| 4261 | gaggcaggga gctctgggag gtggagtccc caccgctttc tctactaggc tcctcctgtt |
| 4321 | ccccaggctt ggggagcttt gcacaaggag actgccccca gcctagtggc acctacctca |
| 4381 | tgggctctgg ggcaggtagg ggaagggcca gtccagctct ggtaatgctg gggggaggca |
| 4441 | taccaaagaa tccaggggca gggagtgggg agggtgactt ccgagctggc ctctcccctt |
| 4501 | cctctaccca gactggggct gggatcctct cctcccgctg taaccatttc tacctcattt |
| 4561 | tgctgcgtgt tgtacatgga cgtatttatc tcctgtctga cgatgctctg cagttgtggt |
| 4621 | ctgtctacct cagaagagac tgtattttaa aagaaagtat tacacagtat taaagcgatg |
| 4681 | acatgtggtt tgcaaaaaaa aaaaaaaaaa a. |

In an embodiment, the FL2 protein sequence comprises:

(SEQ ID NO: 2)
MHWTPEHAQPLNQWPEQHLDVSSTTPSPAHKLELPPGGRQRCHYAWAHDD
ISALTASNLLKRYAEKYSGVLDSPYERPALGGYSDASFLNGAKGDPEPWP
GPEPPYPLASLHEGLPGTKSGGGGGSGALGGSPVLAGNLPEPLYAGNACG
GPSAAPEYAAGYGGGYLAPGYCAQTGAALPPPPPAALLQPPPPPGYGPSA
PLYNYPAGGYAAQPGYGALPPPPGPPPAPYLTPGLPAPTPLPAPAPPTAY
GFPTAAPGAESGLSLKRKAADEGPEGRYRKYAYEPAKAPVADGASYPAAD
NGECRGNGFRAKPPGAAEEASGKYGGGVPLKVLGSPVYGPQLEPFEKFPE
RAPAPRGGFAVPSGETPKGVDPGALELVTSKMVDCGPPVQWADVAGQGAL
KAALEEELVWPLLRPPAYPGSLRPPRTVLLFGPRGAGKALLGRCLATQLG
ATLLRLRGATLAAPGAAEGARLLQAAFAAARCRPPSVLLISELEALLPAR
DDGAAAGGALQVPLLACLDGGCGAGADGVLVVGTTSRPAALDEATRRRFS
LRFYVALPDSPARGQILQRALAQQGCALSERELAALVQGTQGFSGGELGQ
LCQQAAAGAGLPGLQRPLSYKDLEAALAKVGPRASAKELDSFVEWDKMYG
SGH.

In an embodiment, the FL2 is naturally occurring variant having 95% or greater identity with NCBI Reference Sequence: NM_001013690.4 (SEQ ID NO:1). In an embodiment, the FL2 is naturally occurring variant having 96% or greater identity with NCBI Reference Sequence: NM_001013690.4 (SEQ ID NO:1). In an embodiment, the FL2 is naturally occurring variant having 97% or greater identity with NCBI Reference Sequence: NM_001013690.4 (SEQ ID NO:1). In an embodiment, the FL2 is naturally occurring variant having 98% or greater identity with NCBI Reference Sequence: NM_001013690.4 (SEQ ID NO:1). In an embodiment, the FL2 is naturally occurring variant having 99% or greater identity with NCBI Reference Sequence: NM_001013690.4 (SEQ ID NO:1).

In embodiments, the siRNA comprise one of the following pairs of sense/antisense sequences:

| (SEQ ID NO: 3) | |
|---|---|
| Sense: | UUACACAGUAUUAAAGCGAUU |
| (SEQ ID NO: 4) | |
| Antisense: | 5′ UCGCUUUAAUACUGUGUAAUU; |
| or | |
| (SEQ ID NO: 5) | |
| Sense: | CAUCUGAAACCUAGGGUCUUU |
| (SEQ ID NO: 6) | |
| Antisense: | 5′ AGACCCUAGGUUUCAGAUGUU; |
| or | |
| (SEQ ID NO: 7) | |
| Sense: | GUGACUUAUGCUAGGAGGAUU |
| (SEQ ID NO: 8) | |
| Antisense: | 5′ UCCUCCUAGCAUAAGUCACUU; |
| or | |
| (SEQ ID NO: 9) | |
| Sense: | GGUCAGAAGCAGAAUGUAUUU |
| (SEQ ID NO: 10) | |
| Antisense: | 5′ AUACAUUCUGCUUCUGACCUU. |

In an embodiment, the siRNA is double-stranded and comprises SEQ ID NO:3 and 4; SEQ ID NO:5 and 6; SEQ ID NO:7 and 8; or SEQ ID NO:9 and 10.

In an embodiment, the 5′ terminal residue of a strand of the siRNA is phosphorylated. In an embodiment the 5′ terminal residue of the antisense strand of the siRNA is phosphorylated. In an embodiment, the 5′ terminal residue of a strand of the siRNA is not phosphorylated. In an embodiment the 5′ terminal residue of the antisense strand of the siRNA is not phosphorylated.

In an embodiment the inhibitor of Fidgetin-like 2 is provided in a vehicle suitable for application to the skin. In an embodiment the inhibitor of Fidgetin-like 2 is provided in a dermatologically acceptable carrier.

In an embodiment the inhibitor of Fidgetin-like 2 is provided in a bulk-eroding system such as polylactic acid and glycolic acid (PLGA) copolymer based microspheres or microcapsules systems containing the inhibitor of Fidgetin-like 2. In an embodiment, blends of PLGA:ethylcellulose systems may be used as an appropriate carrier. A further medicament in accordance with this aspect of the invention may be formulated in a surface-eroding system wherein the inhibitor of Fidgetin-like 2 is embedded in an erodible matrix such as the poly(ortho) ester and polyanhydride matrices wherein the hydrolysis of the polymer is rapid. A medicament in accordance with this aspect of the invention may also be formulated by combining a pulsatile delivery system as described above and an immediate release system such as a lyophilized injectable composition described above.

The inhibitor may be used in a composition with additives. Examples of suitable additives are sodium alginate, as a gelatinizing agent for preparing a suitable base, or cellulose derivatives, such as guar or xanthan gum, inorganic gelatinizing agents, such as aluminum hydroxide or bentonites (termed thixotropic gel-formers), polyacrylic acid derivatives, such as Carbopol®, polyvinylpyrrolidone, microcrystalline cellulose and carboxymethylcellulose. Amphiphilic low molecular weight and higher molecular weight compounds, and also phospholipids, are also suitable. The gels can be present either as water-based hydrogels or as hydrophobic organogels, for example based on mixtures of low and high molecular weight paraffin hydrocarbons and vaseline. The hydrophilic organogels can be prepared, for example, on the basis of high molecular weight polyethylene glycols. These gelatinous forms are washable. Hydrophobic organogels are also suitable. Hydrophobic additives, such as petroleum jelly, wax, oleyl alcohol, propylene glycol monostearate and/or propylene glycol monopalmitostearate, in particular isopropyl myristate can be included. In an embodiment the inhibitor is in a composition comprising one or more dyes, for example yellow and/or red iron oxide and/or titanium dioxide for the purpose of matching as regards color. Compositions may be in any suitable form including gels, lotions, balms, pastes, sprays, powders, bandages, wound dressing, emulsions, creams and ointments of the mixed-phase or amphiphilic emulsion systems (oil/water-water/oil mixed phase), liposomes and transfersomes or plasters/band aid-type coverings. Emulsifiers which can be employed in compositions comprising the inhibitor of Fidgetin-like 2 include anionic, cationic or neutral surfactants, for example alkali metal soaps, metal soaps, amine soaps, sulphurated and sulphonated compounds, invert soaps, higher fatty alcohols, partial fatty acid esters of sorbitan and polyoxyethylene sorbitan, e.g. lanette types, wool wax, lanolin or other synthetic products for preparing the oil/water and/or water/oil emulsions.

Compositions comprising the inhibitor of Fidgetin-like 2 can also comprise vaseline, natural or synthetic waxes, fatty acids, fatty alcohols, fatty acid esters, for example as monoglycerides, diglycerides or triglycerides, paraffin oil or vegetable oils, hydrogenated castor oil or coconut oil, hog fat, synthetic fats (for example based on caprylic acid, capric acid, lauric acid or stearic acid, such as Softisan®), or triglyceride mixtures, such as Miglyol®, can be used as lipids, in the form of fatty and/or oleaginous and/or waxy components for preparing the ointments, creams or emulsions of the compositions comprising the inhibitor of fidgetin-like 2 used in the methods described herein.

Osmotically active acids and alkaline solutions, for example hydrochloric acid, citric acid, sodium hydroxide solution, potassium hydroxide solution, sodium hydrogen carbonate, may also be ingredients of the compositions and, in addition, buffer systems, such as citrate, phosphate, tris buffer or triethanolamine, for adjusting the pH. It is possible to add preservatives as well, such as methyl benzoate or propyl benzoate (parabens) or sorbic acid, for increasing the stability.

Pastes, powders and solutions are additional forms of compositions comprising the inhibitor of Fidgetin-like 2 which can be applied topically. As consistency-imparting bases, the pastes frequently contain hydrophobic and hydrophilic auxiliary substances, preferably, however, hydrophobic auxiliary substances containing a very high proportion of solids. In order to increase dispersity, and also flowability and slipperiness, and also to prevent agglomerates, the powders or topically applicable powders can, for example, contain starch species, such as wheat or rice starch, flame-dispersed silicon dioxide or siliceous earth, which also serve as diluent.

In an embodiment, the compositions of the invention comprise further active ingredients suitable for stimulating hair growth. In an embodiment, compositions of the invention do not comprise further active ingredients suitable for stimulating hair growth.

In an embodiment of the methods and compositions described herein the subject is a mammal. In an embodiment the subject is human.

In some embodiments, excluded from the present invention is a method performed on skin which has a wound in the area of the skin being treated, i.e. a gross break or discontinuity in the structure of the skin tissue. Examples of wounds include ulcerations, bedsores, grazes, tears, cuts, and punctures. In some embodiments, excluded from the present invention is a method performed on skin which has been subjected to cosmetic laser treatment.

In an embodiment, the cytokeratin 14 is human. In an embodiment, the cytokeratin 14 has the following sequence:

(SEQ ID NO: 11)
MTTCSRQFTS SSSMKGSCGI GGGIGGGSSR ISSVLAGGSC
RAPSTYGGGL SVSSSRFSSG GACGLGGGYG GGFSSSSSSF
GSGFGGGYGG GLGAGLGGGF GGGFAGGDGL LVGSEKVTMQ
NLNDRLASYL DKVRALEEAN ADLEVKIRDW YQRQRPAEIK
DYSPYFKTIE DLRNKILTAT VDNANVLLQI DNARLAADDF
RTKYETELNL RMSVEADING LRRVLDELTL ARADLEMQIE
SLKEELAYLK KNHEEEMNAL RGQVGGDVNV EMDAAPGVDL
SRILNEMRDQ YEKMAEKNRK DAEEWFFTKT EELNREVATN
SELVQSGKSE ISELRRTMQN LEIELQSQLS MKASLENSLE
ETKGRYCMQL AQIQEMIGSV EEQLAQLRCE MEQQNQEYKI
LLDVKTRLEQ EIATYRRLLE GEDAHLSSSQ FSSGSQSSRD
VTSSSRQIRT KVMDVHDGKV VSTHEQVLRT KN

In an embodiment, the subject has experienced hair loss caused by androgenetic alopecia. In an embodiment, the subject has experienced hair loss caused by alopecia areata. In an embodiment, the subject has experienced hair loss caused by telogen effluvium. In an embodiment, the subject has experienced hair loss caused by ringworm. In an embodiment, the subject has experienced hair loss caused by scarring alopecia. In an embodiment, the subject has not experienced scarring alopecia. In an embodiment, the subject has experienced hair loss caused by cosmetic overprocessing. In an embodiment, the subject has not experienced hair loss caused by cosmetic overprocessing.

A shampoo composition is provided comprising (i) an amount of a siRNA or shRNA directed against a DNA or RNA encoding a human Fidgetin like-2, as described herein, effective to enhance hair follicle growth in skin and (ii) a surfactant.

In embodiments, the shampoo composition comprises one or more of sodium lauryl sulfate, sodium laureth sulfate, and cocamidopropyl betaine.

A composition is provided comprising (i) an amount of siRNA or shRNA is directed against an DNA encoding the human Fidgetin-like 2 effective to enhance hair follicle growth in skin contained (ii) in a microneedle array.

In embodiments, the microneedle array comprises a structure made of one or more of dextran, hyaluronic acid and PVP.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

Example 1

Initially, inhibition of FL2 is performed in mouse model where a portion of the skin has been removed or trated to effect removal of hair follicles. The skin is subsequently treated at one location with siRNA or shRNA directed to FL2 and at a second location with control. The skin is then examined at the FL2 treatment site and control site at 8 days or 14 days subsequent to treating.

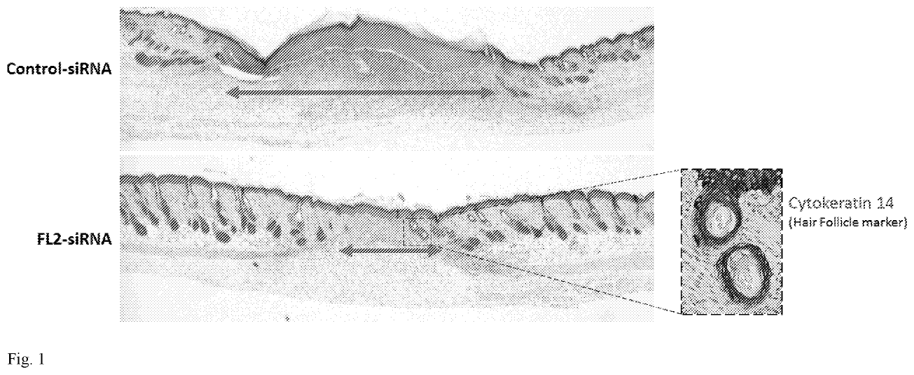

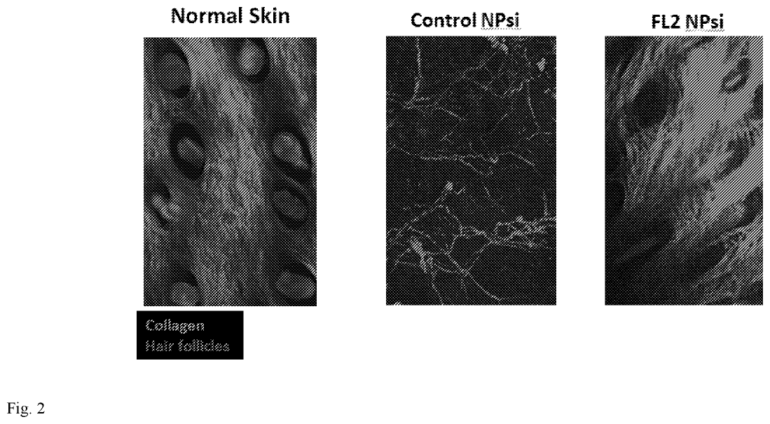

Skin treated with FL2 siRNA in a nanoparticle-encapsulated form (e.g. poloaxomer nanoparticle) showed increased hair follicle numbers and groeth (and cytokertin 14 staining) compared to control. (See FIGS. 1 and 2).

### Example 2

A visibly hairless portion of skin on a human, which previously during the life of the human had exhibited hair at some point, is treated with a topically applied siRNA or shRNA which inhibits Fidgetin-like 2. The topically applied siRNA or shRNA is effective to increase the rate and extent of hair follicle growth in the skin compared to control, and subsequently produces hair.

### Example 3

A visibly hairless portion of skin on a human, which previously at some point during the life of the human had exhibited hair, is treated with a topically applied siRNA or shRNA which inhibits Fidgetin-like 2. The topically applied siRNA or shRNA is effective to increase Cytokeratin 14 expression in the skin portion treated.

### Example 4

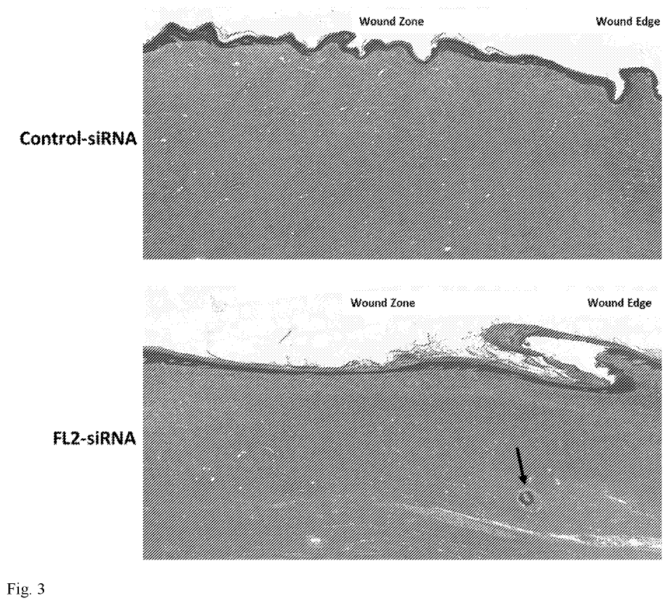

FL2 siRNA stimulates the restoration of hair follicles to burn wound sites: Pigs were administered anesthesia and subjected to third degree thermal burns and then topically treated with FL2-siRNA. Following sacrifice on day 56, wounds were sectioned, stained with H&E and examined for adnexal structures. Hair follicles (indicated by the black arrow in FIG. 3) were noted within the wound zone in FL2 siRNA-treated conditions, while none were present in controls. FL2 siRNA effected hair follicle growth.

What is claimed is:

1. A method of enhancing hair follicle growth in skin comprising directly administering to the skin an amount of a siRNA or shRNA directed against a DNA or RNA encoding a human Fidgetin like-2 effective to enhance hair follicle growth in skin.

2. A method for increasing hair growth in skin comprising directly administering to the skin an amount of a siRNA or shRNA directed against a DNA or RNA encoding a human Fidgetin like-2 effective to increase hair growth in skin.

3. A method for increasing Cytokeratin 14 expression in skin comprising directly administering to the skin an amount of a siRNA or shRNA directed against a DNA or RNA encoding a human Fidgetin like-2 effective to increase Cytokeratin 14 expression in skin.

4. The method of any of claims 1-3, wherein the Fidgetin like-2 comprises the amino acid set forth in SEQ ID NO:2

5. The method of any of claims 1-4, wherein the siRNA is administered.

6. The method of any of claims 1-4, wherein the shRNA is administered.

7. The method of claim 5, wherein the siRNA directed against a DNA or RNA encoding human Fidgetin-like 2 has at least one 2′ sugar modification.

8. The method of claim 6, wherein the shRNA directed against a DNA or RNA encoding human Fidgetin-like 2 has at least one 2′ sugar modification.

9. The method of any of claims 1-8, wherein the siRNA or shRNA is directed against an mRNA encoding the human Fidgetin-like 2.

10. The method of any of claims 1-9, wherein the siRNA or shRNA is directed against an DNA encoding the human Fidgetin-like 2.

11. The method of any of claim 1-5, 7, or 9-10, wherein the siRNA comprises a sequence set forth in SEQ ID NOS:3, 4, 5, 6, 7, 8, 9, or 10.

12. The method of any of claim 1-5, 7, or 9-11, wherein the siRNA is nanoparticle-encapsulated.

13. The method of any of claim 1-5, 7, or 9-12, wherein the siRNA is poloaxomer nanoparticle-encapsulated.

14. A shampoo composition is provided comprising (i) an amount of a siRNA or shRNA directed against a DNA or RNA encoding a human Fidgetin like-2 effective to enhance hair follicle growth in skin and (ii) a surfactant.

15. The shampoo of claim 14 comprising one or more of sodium lauryl sulfate, sodium laureth sulfate, and cocamidopropyl betaine.

16. A composition comprising (i) an amount of siRNA or shRNA is directed against an DNA encoding the human Fidgetin-like 2 effective to enhance hair follicle growth in skin contained (ii) in a microneedle array.

17. The composition of claim 16, wherein the microneedle array comprises a structure made of one or more of dextran, hyaluronic acid and PVP.
